(19) United States
(12) Patent Application Publication
(10) Pub. No.: US 2020/0054815 A1
(43) Pub. Date: Feb. 20, 2020

(54) Extracting Constituent Molecules from Blood or Other Liquids

(71) Applicant: Yoram PALTI, Haifa (IL)

(72) Inventor: Yoram PALTI, Haifa (IL)

(21) Appl. No.: 16/542,978

(22) Filed: Aug. 16, 2019

Related U.S. Application Data
(60) Provisional application No. 62/719,379, filed on Aug. 17, 2018.

(51) Int. Cl. A61M 1/34; A61M 1/36; A61M 1/16

(52) U.S. Cl. CPC A61M 1/3406; A61M 1/3607; A61M 2230/20; A61M 2205/3334; A61M 1/1605

(57) ABSTRACT

Excess water can be removed from blood by passing the blood through channels that are surrounded by nanotubes with spaces therebetween. Each channel is wide enough for blood to flow through, and the nanotubes are spaced close enough to each other to retain the blood within the channels. Gas passing through the spaces between the nanotubes outside the channels comes into contact with the blood at the outer boundaries of the channels, and the excess water in the blood evaporates into the gas. In other embodiments, an undesirable molecule (e.g., ammonia) can be removed from blood by passing the blood through channels that are surrounded by nanotubes with spaces therebetween. Gas passing through the spaces between the nanotubes outside the channels comes into contact with the blood at the outer boundaries of the channels, and the undesirable molecule in the blood diffuses into the gas.

### CROSS REFERENCE TO RELATED APPLICATIONS

This Application claims the benefit of U.S. Provisional Application 62/719,379 filed Aug. 17, 2018, which is incorporated herein by reference in its entirety.

### FIELD OF THE INVENTION

This application is directed to (1) the exchange of gasses between the ambient air and blood; (2) reducing the volume of a liquid solution by removing some of the solvent while maintaining the solute content intact and/or (3) extracting certain dissolved gases from a liquid while maintaining the rest of the liquid's constituents intact.

### BACKGROUND

1: The main function of the lung is to exchange gasses between the ambient air and the blood. Within this framework O₂ is transferred from the environment to the blood while CO₂ is eliminated from the body. In a normal resting human these processes are associated with an O₂ input of about 200-250 cm³/min and an output of about the same amount of CO₂. This exchange is made through a surface area of 50-100 m² of a 0.5-1 μm thick biological membrane separating the alveolar air from the pulmonary blood. This process is associated with the flow of similar volumes of blood and air—about 5 Liter/min. At the given flow rate the blood is in “contact” with the membrane through which diffusion takes place for a time period of ⅓-⅕ sec. In natural systems such as the lung the gas exchange is achieved by diffusion taking place across a thin biological membrane separating two compartments: the gases in the lung alveoli and the gases contained in the blood of the lung capillaries. The gases in the alveolar compartment are maintained at a composition close to that of ambient air or gas by moving the air or gases in and out of the lungs by respiratory movements. The gas exchange is achieved by diffusion through the surface area of the exchange membrane that is extremely large—about 70 m². The driving force for diffusion of gases into and out of the blood is maintained by a very large blood flow through the lung capillaries.

2: in patients suffering from renal insufficiency or failure and hypervolemia, excess water may accumulate in a patient's blood (e.g., due to excess fluid administration). Failure to remove the excess fluid may result in heart failure, peripheral edema, including pulmonary edema which severely affects pulmonary blood gas exchanges, etc.

3: Ammonia and ammonium are highly toxic and therefore must be eliminated from the body. This is normally done by the liver where the blood dissolved ammonium is enzymatically transformed into urea which is less toxic. One of the main functions of the kidney is to eliminate the urea from the blood. However, in patients suffering from renal insufficiency or kidney failure, urea secretion is insufficient, and the patients must be continuously or frequently connected to an artificial kidney to eliminate the urea as well as other undesired compounds (e.g., ammonia) that accumulate.

### SUMMARY OF THE INVENTION

One aspect of the invention is directed to a first method for removing excess water from blood. The first method comprises providing a plurality of fluid flow channels that are surrounded by hydrophobic nanotubes with diameters between 1 and 100 nm, with spaces between the nanotubes. Each of the channels has an outer boundary, an inflow end, and an outflow end, each of the channels is wide enough for blood to flow through, and the nanotubes are spaced close enough to each other to retain the blood within the channels when the blood is flowing through the channels. The first method also comprises passing the blood through the channels; and passing a gas through the spaces between the nanotubes outside the channels so that the gas comes into contact with the blood at the outer boundaries of the channels until the excess water in the blood evaporates into the gas.

Some instances of the first method further comprise determining whether a sufficient amount of water has been removed; and discontinuing the passing of the blood after a sufficient amount of water has been removed.

In some instances of the first method, the nanotubes are carbon nanotubes. In some instances of the first method, each of the channels has a diameter between 2 and 500 μm. In some instances of the first method, the nanotubes have a diameter between 5 and 20 nm. In some instances of the first method, the nanotubes are spaced on centers that are between 1.5 times the diameter of the nanotubes and 5 times the diameter of the nanotubes.

Another aspect of the invention is directed to a second method for removing excess solvent from a liquid. The second method comprises providing a plurality of fluid flow channels that are surrounded by hydrophobic nanotubes with diameters between 1 and 100 μnm, with spaces between the nanotubes. Each of the channels has an outer boundary, an inflow end, and an outflow end, each of the channels is wide enough for liquid to flow through, and the nanotubes are spaced close enough to each other to retain the liquid within the channels when the liquid is flowing through the channels. The second method also comprises passing the liquid through the channels; and passing a gas through the spaces between the nanotubes outside the channels so that the gas comes into contact with the liquid at the outer boundaries of the channels until the excess solvent in the liquid evaporates into the gas.

Some instances of the second method further comprise determining whether a sufficient amount of solvent has been removed; and discontinuing the passing of the liquid after a sufficient amount of solvent has been removed.

Another aspect of the invention is directed to a first solvent evaporation apparatus. The first apparatus comprises a field of at least one million hydrophobic nanotubes with diameters between 1 and 100 nm with spaces between the nanotubes though which gas can travel, with voids in the field positioned to form a plurality of fluid flow channels, each of which is surrounded by the nanotubes. The channels are wide enough for a liquid to pass through, and the nanotubes adjacent to the channels are spaced close enough to each other to prevent the liquid from escaping the channels. The first apparatus also comprises a gas pathway that passes through spaces between the nanotubes and extends from an input to the field of nanotubes to an output from the field of nanotubes; at least one sensor that generates data indicative of how much solvent has been removed from the liquid; and a controller that processes the data from the at least one sensor.

In some embodiments of the first apparatus, the liquid comprises blood and the solvent comprises water.

In some embodiments of the first apparatus, the liquid comprises blood and the solvent comprises water, and the apparatus further comprises a surface upon which the water condenses and a container for holding the condensed water. In these embodiments, the at least one sensor comprises a water level sensor that generates data indicative of how much water is in the container.

In some embodiments of the first apparatus, the liquid comprises blood and the solvent comprises water, and the at least one sensor comprises (a) a humidity sensor that outputs a first signal indicative of humidity of gas exiting the gas pathway and (h) a flow sensor that outputs a second signal indicative of flow of gas exiting the gas pathway. In these embodiments, the controller determines how much water has exited the gas pathway based on the first signal and the second signal.

Another aspect of the invention is directed to a third method for removing a specific molecule from a liquid. The third method comprises providing a plurality of fluid flow channels that are surrounded by hydrophobic nanotubes with diameters between 1 and 100 nm, with spaces between the nanotubes. Each of the channels has an outer boundary, an inflow end, and an outflow end, each of the channels is wide enough for the liquid to flow through, and the nanotubes are spaced close enough to each other to retain the liquid within the channels when the liquid is flowing through the channels. The third method also comprises passing the liquid through the channels; and passing a gas through the spaces between the nanotubes outside the channels so that the gas comes into contact with the liquid at the outer boundaries of the channels until the specific molecule in the liquid diffuses into the gas.

Some instances of the third method further comprise determining whether a particular amount of the specific molecule has been removed; and discontinuing the passing of the liquid after the particular amount of the specific molecule has been removed.

In some instances of the third method, the liquid is blood and the specific molecule is ammonia. In some instances of the third method, the nanotubes are carbon nanotubes. In some instances of the third method, each of the channels has a diameter between 2 and 500 μm. In some instances of the third method, the nanotubes have a diameter between 5 and 20 nm. In some instances of the third method, the nanotubes are spaced on centers that are between 1.5 times the diameter of the nanotubes and 5 times the diameter of the nanotubes.

Sonic instances of the third method further comprise analyzing the gas that has passed through the spaces between the nanotubes outside the channels to determine whether the specific molecule is present.

Some instances of the third method further comprise analyzing the gas that has passed through the spaces between the nanotubes outside the channels to determine how much of the specific molecule is present.

Another aspect of the invention is directed to a fourth method for introducing a specific molecule into a liquid. The fourth method comprises providing a plurality of fluid flow channels that are surrounded by hydrophobic nanotubes with diameters between 1 and 100 nm, with spaces between the nanotubes. Each of the channels has an outer boundary, an inflow end, and an outflow end, each of the channels is wide enough for the liquid to flow through, and the nanotubes are spaced close enough to each other to retain the liquid within the channels when the liquid is flowing through the channels. The fourth method also comprises passing the liquid through the channels; and passing a gas that includes the specific molecule through the spaces between the nanotubes outside the channels so that the gas comes into contact with the liquid at the outer boundaries of the channels until a desired quantity of the specific molecule diffuses into the liquid.

In some instances of the fourth method, the liquid is blood.

### BRIEF DESCRIPTION OF THE DRAWINGS

FIG. 1 shows scanning electron microscope images of a carpet-like field of vertically aligned carbon nanotubes with voids formed therein.

FIG. 2A is a schematic representation of a gas exchanger that has two gas exchange units of a first type connected in series.

FIG. 2B is a schematic representation of a gas exchanger that has two gas exchange units of a second type connected in series.

FIG. 3A depicts a preferred way to lay out the nanotubes for the FIG. 2B embodiment.

FIG. 3B depicts a preferred way to lay out the nanotubes for the FIG. 2A embodiment.

FIG. 3C depicts another preferred way to lay out the nanotubes for the FIG. 2B embodiment

FIG. 3D is a detailed view of FIG. 3A.

FIG. 4A is a more detailed representation of a single gas exchange unit of the FIG. 2B embodiment.

FIG. 4B is a magnified view of a region of FIG. 4A.

FIG. 5 depicts a gas exchanger with ten gas exchange units connected in parallel.

FIGS. 6A, 6B, and 6C depict three ways how a gas exchanger can be used as an artificial lung.

FIG. 7 is a schematic representation of how a gas exchanger can be used as a respiratory assist device.

FIG. 8 depicts a water-removal apparatus in which the amount of water that is removed from blood by evaporation into a gas is measured directly.

FIG. 9 depicts a water-removal apparatus in which the amount of water that is removed from blood by evaporation into a gas is measured indirectly.

### DESCRIPTION OF THE PREFERRED EMBODIMENTS

A first set of embodiments relate to a Gas Exchanger (“GE”) that will be described here within the framework of an artificial lung for efficient gas exchange (O₂, CO₂, etc.) between compartments such as human (or animal) blood and ambient air or some other gas. More specifically, the first set of embodiments are directed to an artificial lung and respiratory aid based on a structure made of nanotubes.

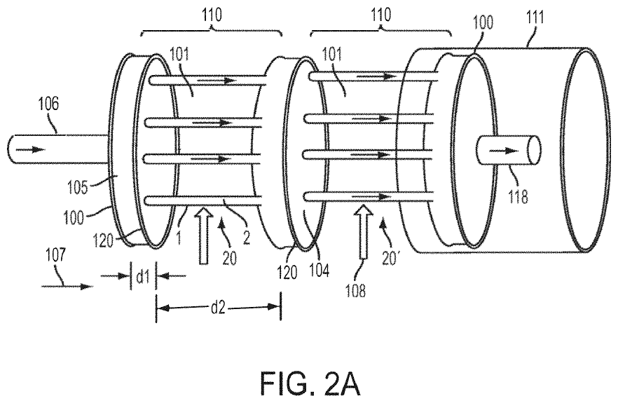

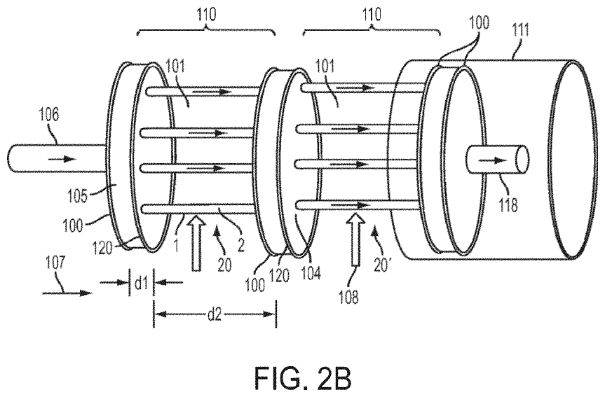

The GE system contains one or more gas exchange units 110 (GEU), and FIG. 2A is a schematic representation of two such GEUs connected in series. Each GEU 110 includes a matrix of parallel aligned blood flow channels 2 (“BFC”, also referred to herein as “fluid flow channels”). FIG. 2A schematically depicts a first set 20 of four parallel BFCs in one GEU on the left, and a second set 20′ of four parallel BFCs in a second GEU on the right, with the first GEU connected in series with the second GEU. Note that while FIG. 2A schematically depicts only four parallel BFCs in each GEU, in practice there will be many more BFCs in each GEU. For example, if the BFCs are 20 μm in diameter and are spaced on 40 μm centers, 62,500 BFCs would fit in a 1 cm² area. Note also that while FIG. 2A depicts two GEUs in series, that number may vary, and a given GE could have more than two GEUs in series, or only a single GEU. In alternative embodiments, a plurality of GEUs may be connected in parallel instead of in series.

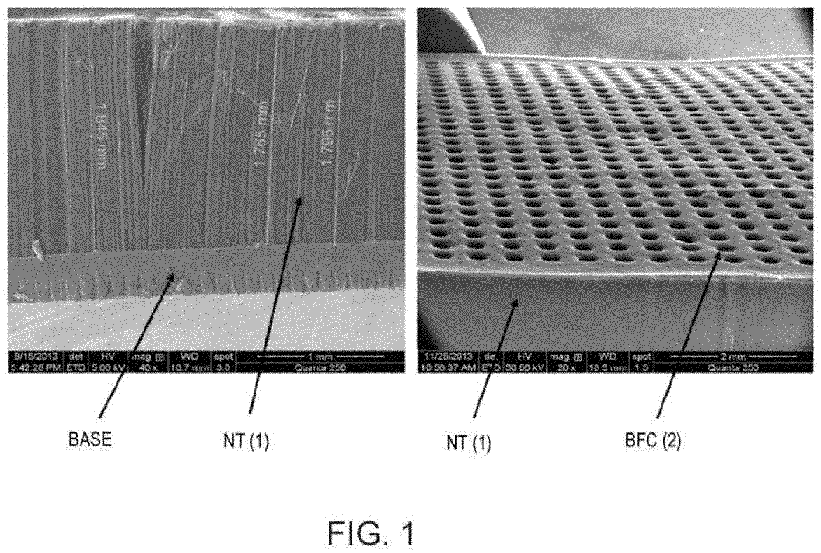

The GE utilizes a plurality of hydrophobic nanotubes (NTs), e.g., carbon nanotubes. The NTs are highly hydrophobic, and the overall plurality of nanotubes may be referred to as a nanotuhe “field.” The NTs may be free standing; held together by Van der Waals forces; or mounted on a base made of, e.g., alumina, silicon, etc. Optionally, the structural integrity of the NTs can be enhanced by coating and infiltrating the NTs with an agent such as carbon (e.g., using well-known processes for forming vertically aligned carbon nanotubes). The field of NTs includes a large number of blood flow channels (BFCs) (“channels”) formed as discrete voids within the field of NTs. The field of NTs and the BFCs 2 therein may be constructed as described in U.S. Pat. Nos. 9,138,522 and 9,827,534, each of which is incorporated by reference in its entirety. FIG. 1 shows scanning electron microscope images of a carpet-like field of vertically aligned carbon nanotubes with voids formed therein that act as the BFCs.

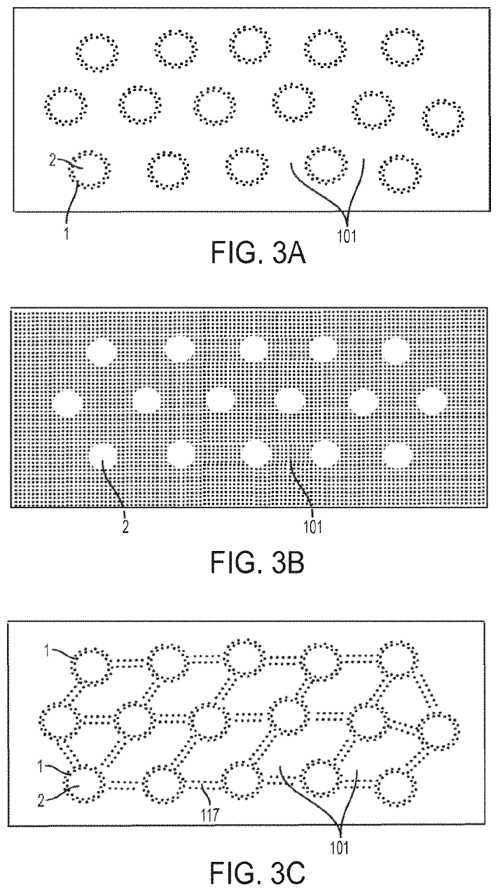

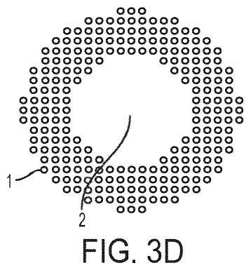

Each BFC is surrounded by NTs, which are shown in FIG. 3B (but not shown in FIG. 2A). FIG. 3B depicts a preferred way to lay out the NTs to define the BFCs for the FIG. 2A embodiment, with the NTs laid out in a field pattern. The view depicted in FIG. 3B is a cross section through the BFCs and the NTs, and there are voids in the field of NTs that define the BFCs 2. In some embodiments, the diameter of the voids is between 2 and 500 μm, and in some embodiments the diameter is between 5 and 20 μm. (Note that all the figures in this application are not drawn to scale). In some preferred embodiments, the NTs within the field (i.e., outside the voids) are arranged as a two dimensional matrix. In some embodiments, the NTs have diameters on the order of 1-100 nm, more preferably between 5 and 20 nm, and still more preferably between 10 and 20 nm. In other embodiments, the NTs have diameters between 2-10 nm. In some preferred embodiments, the distances between the centers of the NTs is from 20-500 nm, and in some preferred embodiments, the distance between the centers is 100-300 nm. In some preferred embodiments, the height of the NTs is 1-2 mm.

The optimum distance between the NT centers will be related to the NT diameter, so that the NTs do not end up too far away from each other. More specifically, when thinner NTs are used, the NTs should preferably be packed more closely together. Preferably, the spacing between NTs will be not more than a few diameters of the NTs, and will more preferably be on the order of 1 diameter. For example, if NTs with 10 nm diameter are used, the NTs would preferably be spaced on centers of about 20 nm, which would mean that the spacing between adjacent NTs would be around one diameter. But if NTs with 20 nm diameter are used, the NTs would preferably be spaced further apart, on centers of about 40 nm. A suitable relationship between the NT diameter and the NT spacing is to space the NTs on centers that are between 1.5 times the diameter of the NT and 5 times the diameter of the NT. For example, if NTs with a diameter of 10 nm are used, the NTs should preferably be spaced on centers between 15 and 50 nm. In less preferred embodiments, the NTs are spaced centers between 1 times and 10 times the diameter of the NTs, or even between 0.5 times and 20 times the diameter of the NTs. Note that the NT packing or density affects the resistance to flow of the gas through the “forest” or “field” of NTs, which is an additional consideration that may be adjusted depending on the specific need. Note that the density of the NTs as well as the BFCs determine both (a) the exchange capacity and (b) the resistance to gas flow, and both of these parameters should be considered in selecting the layout and spacing of the NTs.

Methods for fabricating large masses of parallel carbon NTs, as depicted in FIG. 1B, were described by Li et al. in Highly-Ordered Carbon Nanotube Arrays for Electronics Applications, Applied Physics Letters (1999); 75, 367-369. The desired placement of the NTs can be achieved by positioning the NTs at the desired locations using standard techniques. For example, the NTs may be fabricated on a substrate (which serves as a NT base) at the desired position using a lithography-based process. This may be accomplished by depositing catalysts on a substrate that has been masked to create the desired pattern, and then exposing it to carbon gas. The carbon from the gas then forms NTs (by self-assembly) on the spots where the catalyst has been deposited. NTs will not grow on the other parts of the substrate.

Collectively, the substrate, the field of NTs positioned on the substrate, and the voids within that field form a gas exchange “plate,” and this plate is used as a building block in the system described below. Each of the plates is formed from a very large number (e.g., millions or billions) of hydrophobic NTs with diameters between 1 and 100 nm. In some preferred embodiments, the NTs are vertically aligned carbon nanotubes (which are highly hydrophobic) that remain attached to the substrate on which they were formed. The NTs are positioned in a “field” with a large number (e.g., thousands or hundreds of thousands) of voids in that field that define vertical channels through which blood can pass. These channels are referred to herein as “blood-flow channels,” and the substrate has a hole that aligns with each of these blood-flow channels.

In alternative embodiments, each of these plates may be formed from a very large number (e.g., millions or billions) of interconnected NTs, with interconnections between the NTs that are sufficient to hold the plate together without requiring a substrate (in which case the substrate on which the NTs are originally grown can be removed). Examples of this variety of plate are described in “c-VACNT™ Enabled Fluid Reactor Innovations” by K. Strobl et al. (June 2019); “Vertically aligned carbon nanotube arrays as a thermal interface material” by L. Ping et al., APL Mater. 7, 020902 (2019); doi: 10.1063/1.5083868 (Feb. 2019); and in “Transfer of vertically aligned carbon nanotube arrays onto flexible substrates for gecko-inspired dry adhesive application” by Yang Li et al., RSC Advances, Issue 58 (May 2015). As in the previous variation, when this type of gas-exchange plate is used, a large number (e.g., thousands or hundreds of thousands) of preferably identical vertical blood-flow channels pass through the field of NTs.

The blood-flow channels are wide enough (e.g., between 2 and 500 μm) for the blood to flow through, and the NTs are spaced close enough together to retain the blood within the blood-flow channels, due to the hydrophobic nature of the NTs and the surface tension of the blood.

Returning to FIG. 2A, blood flows through the depicted device from left to right, in the blood flow direction 107. Blood that originates from the person's blood circulation flows through the Inflow channel 106 into an initial blood pool 105 that is bounded by a support 100 on the left, by the first NT base 120 on the right, and by casing 111 in directions that are perpendicular to the blood flow direction 107. In alternative embodiments, the boundary of the blood pool in directions that are perpendicular to the blood flow direction 107 can be implemented using an appropriate ring enclosure. The width of the initial blood pool is d1, and a suitable dimension for d1 is between 0.1-4 mm. However, any distance d1 that permits blood flow without adding a significant resistance to flow can be used instead.

The NT base 120 is preferably the substrate on which the NTs that surround the BFCs were fabricated, and the NT base 120 should have a hole or perforation 104 located at the center of each BFC. The NTs extend to the right from the NT base 120 and span a distance d2 to define the BFCs, which are oriented parallel to the direction of blood flow 107 and perpendicular to the gas flow direction 108. In some preferred embodiments, the distance d2 is between 0.1-1 cm. Because the NTs are grown on the NT base 120 and remain attached to it, no leakage near the base is expected. The NTs are held firmly in place by the extremely strong Van der Waals forces characterizing such nm scale structures. As a result of this configuration, blood that flows into the pool 105 will flow to the right through the perforations 104 in the NT base 120 and continue towards the right into and through the first set 20 of BFCs 2 in the first GEU.

A second NT base 120 is preferably positioned a short distance (e.g., between 0.1-4 mm in some embodiment or between 0.5 and 2 mm in some embodiments) away from the right end of the NTs that define the first set 20 of BFCs 2. When blood exits the first set of BFCs, it will flow into the gap between (a) the right end of the NTs that define the first set 20 of BFCs 2 and (b) the second NT base 120. The second GEU has a second set 20′ of BFCs 2 that is similar in construction to the first set 20 of BFCs 2, each BFC having an aligned perforation 104 in the NT base. The blood that enters the gap will then flow to the right through the perforations 104 in the second NT base 120 and continue towards the right, into and through the second set 20′ of BFCs 2 in the second GEU.

Note that when the blood exits the first set 20 of BFCs 2 and flows into the gap, surface tension of the blood (which is a water-based liquid) together with the hydrophobicity of the carbon NTs should prevent the blood from backing up into the very small spaces between the NTs that form the first set 20 of BFCs 2. Instead, the blood should flow to the right into the second set 20′ of BFCs 2 in the second GEU, because the diameter of the BFCs in the second GEU is orders of magnitude larger than the very small spaces between the NTs in the first GEU. The blood would then flow according to the pressure gradient through the second GEU (i.e., in the blood flow direction 107 through the holes in the second NT base 120 and then through the second set 20′ of BFCs 2 in the second GEU) rather than backwards. Note that the distance between adjacent NTs (i.e., less than a few diameters of the NTs, and preferably on the order of 1 diameter) is low enough to prevent blood plasma (or water) from penetrating the space between the NTs due to surface tension.

In alternative embodiments, additional stages (not shown) may be added in series. The blood eventually reaches the last GEU. A final support 100 is preferably positioned a short distance (e.g., between 0.1-4 mm in some embodiments, or between 0.5 and 2 mm in some embodiments) away from the right end of the NTs that define the last set 20′ of BFCs 2. When blood exits the last set of BFCs, it will flow into the gap between (a) the right end of the NTs that define the last set 20′ of BFCs 2 and (b) the final support 100. From there it will flow into the blood outflow channel 118.

While the blood is in the BFCs 2 in any of the stages, the blood has a chance to interact with the gases in the gas flow region 101. These gases flow in a gas flow direction 108 (i.e., up in FIG. 2A) that is preferably perpendicular to the direction of blood flow 107 (i.e., to the right in FIG. 2A). At the end of this process the blood continues through outflow channel 118 back to the blood circulation.

It is important note that, regardless of which variety of plate is used, the BFCs 2 have no coating or membrane to keep the blood from escaping the BFC. However, due to the high density (i.e., the close spacing) of the hydrophobic NTs surrounding the BFCs and the high surface tension of water, when a water-based fluid, such as blood, occupies or flows in the BFC, it will not leak out of the BFCs into the gas flow region 101. In other words, the NTs surrounding the BFC 2 form a virtual boundary for the liquid flow. The interactions between the blood and the gas occurs at this virtual boundary.

In addition, regardless of which variety of plate is used, the blood will travel through the blood-flow channels, while the gas that will exchange molecules with the blood permeates the spaces between the NTs (analogous to the way air permeates through a forest of trees). Because the NTs in the field are relatively densely packed, they can present significant resistance to horizontal flow of gas. So to ensure that the gas reaches the blood-flow channels, conduits that are free of NTs may optionally be included in the plate in some embodiments. In these embodiments, gas will permeate to the boundaries of the blood-flow channels by the combination of gas flowing through the conduits and diffusion from the conduits to nearby blood-flow channels.

Casing 111, a rigid biocompatible housing, seals the initial Blood Pool 105 as well as the one or more GEUs 110 contained within the casing 111. This permits gas exchange between the blood in the BFC and the air (or other gases) in the gas flow regions 101.

FIG. 2B depicts an alternative embodiment that is similar to the FIG. 2A embodiment, except that additional blood pools 105 are added between adjacent GEU stages. In this embodiment, blood exiting one GEU is collected into a blood pool 105 confined between a planar support 100 (on the left) and the subsequent NT base 120 before it enters the next GEU. The planar support 100 for each GUI stage has holes or perforations 104 that are aligned to the position of the BFCs 2 of the previous stage GEU (except for the input of the first stage and the output of the last stage, which preferably each have a single larger port). For any given stage, the distance between the planar support 100 and the subsequent NT base 120 is d1, and a suitable dimension for d1 is between 0.1-4 mm. However, any separation that permits blood flow without adding a significant resistance to flow can be used instead. Casing 111, a rigid biocompatible housing, seals all the Blood Pools 105 as well as all the GEUs 110 contained within the casing 111.

In this FIG. 2B embodiment, the NTs may be laid out as shown in FIG. 3B, which is discussed above. But alternative layouts for the NTs may also be used in this embodiment.

FIG. 3A depicts a first alternative approach for laying out the NTs to define the BFCs in the FIG. 2B embodiment. In this approach, the NTs are laid out in pattern of rings 1 so that the inner boundary of each ring 1 defines a BFC 2. The depicted view is a cross section through the BFCs and the NTs. The diameter of the inner boundary of the ring is between 2 and 500 μm in some embodiments, and between 5 and 20 μm in some embodiments. In this approach, the thickness of each ring (i.e., the distance between the innermost NTs of the ring and the outermost NTs of the ring) is preferably between 100 nm and 10 μm, and the NTs within the ring are preferably spaced on centers between 10 and 100 nm. As in the FIG. 3B approach, the distance between the NT centers is preferably related to the NT diameter, so that the NT do not end up too far away from each other. FIG. 3D is a detailed view of a ring 1 and the BFC 2 of FIG. 3A. The NTs in the ring 1 may be laid out in a two dimensional matrix, as shown in FIG. 3D, or in any other layout that maintains appropriate spacing between the centers of the NTs.

FIG. 3C depicts a second alternative approach to lay out the NTs to define the BFCs in the FIG. 2B embodiment. The depicted view is a cross section through the BFCs and the NTs. This approach is similar to the approach depicted in FIG. 3A, except that additional NTs are added to provide structural support. The additional NTs may be configured to form support bridges 117, as shown in FIG. 3C, but alternative layouts for the additional NTs may be used instead. Examples of such alternative layouts (not shown) include stripes and grids. The layout of the additional NTs may be selected to provide structural strength without unduly increasing the resistance to air flow. Another example (not shown) would be to add clusters of NTs at midpoints between adjacent BFCs, arranged in a column-like fashion to add structural support. For example, a set of NTs arranged to fill in a circle with a diameter of 10 μm, with the NTs in the set spaced on centers between 10 and 100 nm, could serves as a support column. Each NT in such a support column would have the same length d2 as the NTs in the rings that surround the BFCs. Note that in these configurations (e.g., FIGS. 3A and 3C), the resistance to air flow through the field of NTs is lower than in the FIG. 3B embodiment without compromising the gas exchange capabilities.

For all of the embodiments described above, the blood in the inflow channel 106 is preferably venous blood that is low in oxygen and rich in CO₂. The two blood gases undergo an exchange with the gas flowing in the gas flow region 101 around the BFCs in a direction 108 that is preferably normal to that of the BFC blood flow 107. This incoming gas is preferably rich in oxygen and has a low or zero concentration of CO₂ so that the gas exchange is by diffusion along the concentration gradients. The blood in the outflow channel 118 will then be richer in O₂ than the incoming blood.

The efficacy of the gas exchange is a function of the area of contact between the flowing blood and the flowing gas that may be oxygen or air. As mentioned above, in a normal pair of lungs this contact surface area is typically about 70 m² while the blood flow is 5-7 L/min and air flow is similar. The amount of Oxygen or CO₂ exchanged in normal human lungs is typically 200-250 cm³/min.

Let us now compute the parameters of gas exchange that satisfy the normal physiological requirements: The total BFC surface area that is needed for the gas exchange is a direct function of the BFC diameter and packing, i.e. the distance between the BFCs, and the total number of BFCs in the GE volume. For a GE having a total volume of 2 liters (e.g., 10 cm×10 cm×20 cm), the surface area available for exchange is independent of the arrangement of the GEUs within the GE, i.e. in series or in parallel, or their spatial configuration. For such a GE, if we assume that the BFC Radius is 10 μm, and the center-to-center distance of the BFCs is 40 μm, the total gas-blood exchange area is close about 80 m², which is approximately equal to a typical pair of lungs. The Diffusion Capacity will therefore be over 2000 cm³ O₂ per min (which exceeds the requirement of 250 cm³/min), and the Blood volume will be about 400 cm³ (which is comparable to that of the adult human respiratory system).

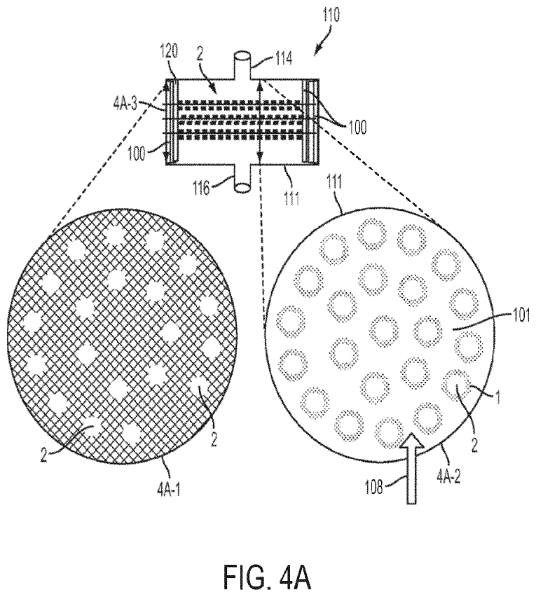

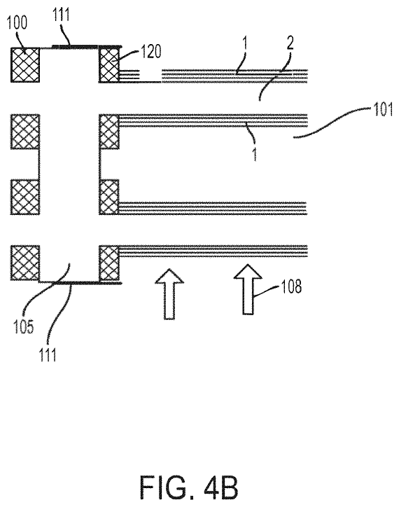

FIG. 4A is a more detailed representation of a single GEU 110 of the FIG. 2B variety, in which the NTs are arranged in rings 1 (as shown in FIG. 3A and 3D). The GEU 110 has a set of parallel BFCs located between a first support 100 and a first NT base 120 on the left and a pair of supports 100 on the right. The O₂ rich gas flows into the gas inlet 116, flows past the BFCs 2, and exits the gas outlet 114. As the gas flows past the BFCs 2, it comes in contact with the blood in the BFCs so that gases can be exchanged. 4A-1 is a cross section through the first support 100, which shows the holes in the support, and 4A-2 is a cross section through a set of BFCs 2. The holes in the NT base 120 line up with the BFCs, as best seen in FIG. 4B, which is a magnified view of the region 4A-3 of FIG. 4A. The holes in the support 100 also line up with the BFCs of the previous stage, as best seen in FIG. 4B. Note that although FIG. 4A schematically shows only 22 BFCs, there will in fact be many more BFCs that are spaced much more closely together, as described above.

The overall GE preferably includes a plurality of GEUs connected together. The GEUs may be connected in series or in parallel to form the GE. Since connecting GEUs in series will increase the flow resistance, the number of GUIs that are connected in series should preferably be limited (e.g., to not more than ten). The GEUs may also be connected in a series/parallel combination. For example, three GEUs may be connected in series, and then the resulting set of three GEUs may be connected in parallel with five similar sets of three series-connected GEUs. Different series/parallel combinations may also be used.

The number of GEUs that are used in any given GE may vary, depending on the required surface area for diffusion. In some embodiments, a GE may contain between 2 and 20 GEUs connected in series, or between 2 and 10 GEUs connected in series.

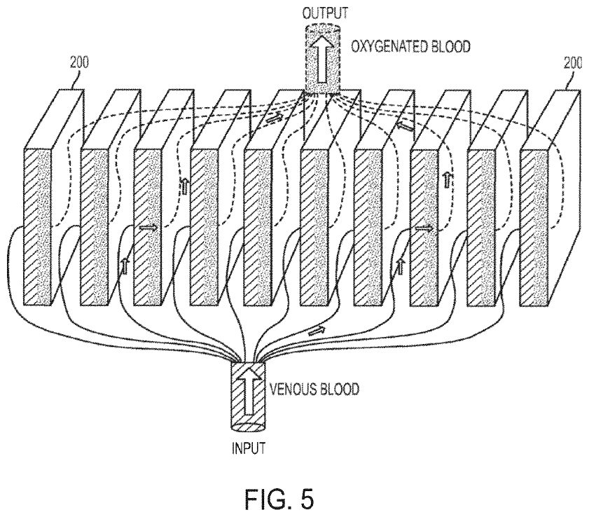

Optionally, a plurality of GEUs may be combined into subsystems, and those subsystems may be connected in series, in parallel, or in series/parallel combinations to form the overall GE. When the BFCs are 20 μm in diameter and are spaced on 40 μm centers, 62,500 BFCs would fit in a 1 cm² area, and would impose resistance to flow through the BFCs of 1.63·10⁵ g/(s cm⁴). One example of suitable dimensions for a subsystem for use in a GE would be a width of 10 cm, a height of 10 cm, and a thickness of about 1.1 cm. The 1.1 cm thickness could be made of 10 GEUs that are each 0.1 cm thick, arranged in series as depicted in FIG. 2A, separated by 9 NT bases 120 that are each 0.1 mm thick between the GEUs, plus an additional blood pool 105 at each end. These 10×10×1.1 cm subsystems can then be configured in parallel to make the complete GE. FIG. 5 depicts ten such subsystems 200 connected in parallel. When 20 such subsystems are arranged in parallel, resistance to flow will be sufficiently low so that less than 50 mmHg is required to induce the required 5-7 L/min blood flow (for subsystems of 10 cm×10 cm×1.1 cm each with the BFC, diameter and spacing described above). The Dwell Time (i.e., the time flowing blood is exposed to gas exchange when flowing from input to output) for this configuration will be over 1 sec, which is well above the required minimal value of 0.2-0.4 sec.

In alternative configurations, the subsystems may be smaller e.g., 2 cm wide, 2 cm high, and about 1 cm thick, with similar internal construction to the 20×20×1.1 cm subsystems described above. These 2×2×1 cm subsystems can then be configured in parallel and/or in series to form the complete GE. In other alternative embodiments, the subsystems may be larger (e.g., 20 cm wide, 20 cm high, and about 2 cm thick).

Yet another possible configuration of GEUs for forming a GE would be to connect multiple (e.g., 2000) 1 cm² units in parallel into a subsystem, and then connect 10 such subsystems in series. In such a GE system, the surface area of oxygen diffusion is sufficient for physiological quiet breathing and the resistance to flow in the BFCs would be only 815 g/(s cm⁴). This configuration would also have a pressure drop of less than 50 mmHg when 5-7 L/min of blood is flowing through the system.

Note that the diffusion capacity of the GEs discussed herein can be even higher than human lungs in which a 0.5-1 μm membrane (made up of living cells and a basal membrane) is interposed between the air and blood. In contrast, there is a direct air-blood contact in the GE. The continuous gas flow around the BFCs in the GE is also more efficient than the in/out air flow in the lungs during natural respiration.

We turn next to the efficacy of the Gas Exchanger with regards to CO₂. The water Diffusion coefficients of CO₂ and O₂ are similar while the solubility of CO₂ is about 24 times higher than that of O₂. As the O₂ and CO₂ concentration difference between oxygenated and reduced blood are similar, the diffusion rate of CO₂ is about 20 times that of O₂. Thus, the CO₂ transport in all the above processes is expected to be superior to that of O₂.

Two examples of clinical applications are using the GE as an artificial lung and using the GE as a respiratory assist device.

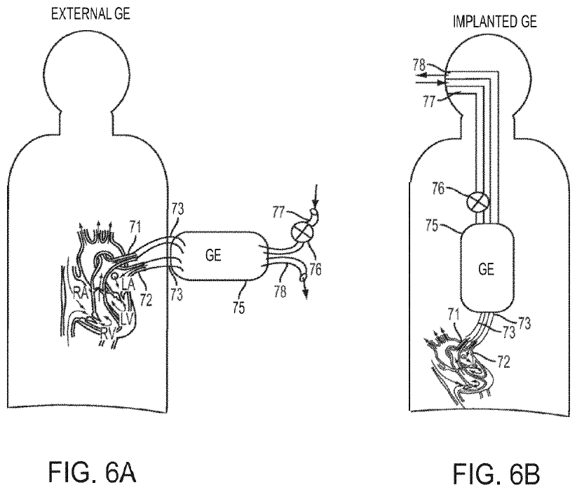

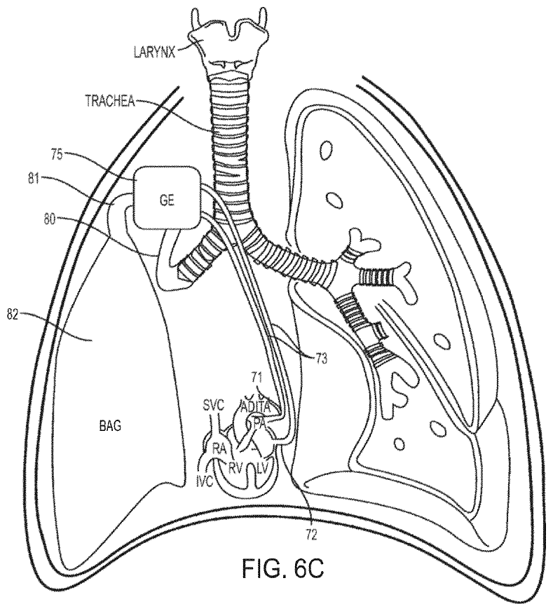

FIGS. 6A, 6B, and 6C depict how a GE 75 can be used as an artificial lung, in which case the GE 75 replaces either one or both lungs. In this application, the GE may be implanted (as shown in FIG. 6B and 6C) or external (as shown in FIG. 6A). In either case, the blood enters the GE 75 via tubing 73 from the pulmonary artery 71 and the blood is returned to the pulmonary vein 72 from the GE via tubing 73. Alternatively, the venous blood source can be from the right atrium or a large vein, and the output can be to the left ventricle. Air or oxygen can be pumped into the GE 75 via the gas input tube 77 by pump 76 and the exhaust leaves via the exhaust tube 78, as shown in FIG. 6A and 6B.

Alternatively, air can be driven through the trachea and main bronchi via natural breathing as shown in FIG. 6C. In this case the air flows in tube 80 from the bronchi into the GE 75 that is connected via tube 81 to an expandable gas bag 82, which inflates and deflates, i.e. changes volume during inspiration and expiration, respectively. Tubes 80 and 81 serve also as the exhaust tubes for the gas exiting the bag 82 via the GE 75 back into the main bronchi and environment.

In any of these embodiments, the blood flow can be maintained by the natural pressure generated by the right ventricle or an appropriate blood vessel. Alternatively it can be driven by an external or implanted pump designed to generate blood flow for long periods of time. Such pumps are commercially available. The blood exiting the GE is returned to the body via a pulmonary vein 72 or veins, or any other appropriate blood vessel.

The flow rates for both blood and air are preferably adjustable to match the needs of the person, etc. this adjustment may be dynamic according to the changing need, for example during exercise. The adjustment may be controlled by sensors of a relevant physiological parameter such as the partial pressure of O₂ and/or CO₂ in the blood, Hb O₂ saturation (oximetry), pH, etc. To supply the O₂ (or other gas) needs, which amount to approximately 250 cm³/min for a resting adult man, a flow of about 5-7 L/min oxygenated blood is required; and this may need to be increased by a factor of up to 4-5 during exercise. An additional factor that should preferably be taken into consideration is the time the flowing blood is exposed to the gas diffusion process, the dwell time. In the normal resting human lung this duration is about ⅓-⅕ of a sec while the flow velocity is usually under 100 cm/s. The blood flow in the GE is compatible with these requirements. When the subject's heart is healthy, the blood flow may be powered by the patient's heart. Note that the series/parallel configuration of GEUs within the GE may be selected in advance to provide a desired flow resistance. To increase the resistance, the number of GEUs connected in series should be increased. To decrease the resistance, the number of GEUs connected in series should be reduced, and the number of parallel connections should be increased.

The corresponding air (or oxygen) flow is also about 5-8 L/min at rest and up to 5 times larger during exercise. When implanted, the Gas inlet 116 and Gas Outlet 114 (shown in FIG. 4) can be connected to the patient's bronchial system as shown in FIG. 6C and flow can be maintained by respiratory movements or a by an appropriate pump. When the GE is external (as shown in FIG. 6A) or implanted without the use of the respiratory ventilation ability (FIG. 6B), the gas Inlet & Outlet are preferably in communication with the ambient air or a gas reservoir through appropriate filters. In this case, gas flow can be continuously driven by an appropriate pump and regulated by appropriate sensors.

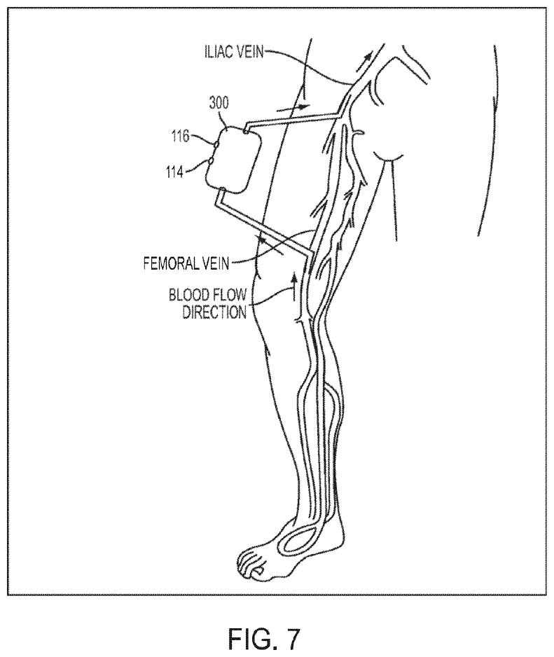

FIG. 7 is a schematic representation of how the GE can be used as a respiratory assist device, in order to provide additional oxygenation of blood for a patient with a failing respiratory system. In these cases the GE 300 is positioned externally, as shown, or implanted. In this application, the blood flowing through the GE is preferably derived here from a large blood vessel, for example the femoral vein. The blood exiting the GE can be introduced back into the femoral vein or veins, or any other appropriate blood vessel.

In certain circumstances, it may he beneficial to remove some of the water, or other liquid, from a solution without affecting the nature of the materials dissolved or suspended in it. For example, when excess water has accumulated in a patient's blood (e.g., in a patient with kidney failure, or in situations where too much fluid is administered to a patient), it would be desirable to remove some or all of the excess water from the patient's blood without affecting the other blood constituents.

A second set of embodiments is directed to a Fluid Reducer system designed to effectively remove water from blood (or other liquid). The hardware configuration of these embodiments is similar to the hardware configuration of the gas exchange are described above in connection with FIGS. 1-5 and 7. For example, in a patient with kidney failure, these embodiments can be connected to a patient's brachial or femoral blood vessel (e.g. as depicted in FIG. 7).

Blood extracted from the patient flows through the BFCs 2, as described above in connection with FIGS. 2-5. Gas entering at Gas inflow is able to flow lengthwise through the field of NTs, from one side of the field to the opposite side of the field, flowing through and restricted to the interstitial spaces between the NTs. The Gas exits through an Outflow port. In addition, liquid blood will fill and flow through the BFCs 2, which extend in the height direction of the field of NTs. Thus, the areas along which the gas flows, through the field of NTs, form at least part of a gas pathway, and the BFCs 2 form at least part of a blood pathway.

This arrangement provides an extremely large surface area for contact and molecular transfer between the blood (in the BFCs 2) and the gas flowing along the gas pathway, i.e., permeating through the field of NTs, and flowing around the virtual boundaries of the BFCs 2. For example, for a field that is 2 mm in height, with BFCs 2 having a radius of 25 microns spaced 25 microns apart, there are about 20,000 BFCs per square centimeter, and the total surface area of these 20,000 BFCs—i.e., area across which water molecules can be transferred to the gas—is about 30 square centimeters.

As explained above, the NTs surrounding the BFCs 2 form a virtual boundary for the blood flow. The interaction between the blood and the gas (e.g., the evaporation of the water from the blood into the gas) occurs at this virtual boundary. When the blood flows in the BFCs, the blood will be in direct contact with the flowing dry gas and some of the water in the blood will evaporate. This evaporation will transfer water molecules from blood in the BFCs 2 to the flowing gas, thereby lowering the water content of the blood in the BFCs. Lowering the water content of the blood (which also reduces blood volume) can be beneficial in a variety of contexts, including but not limited to extracorporeal oxygenation (e.g., ECMO or as described above in connection with the first set of embodiments).

The amount of water that is removed by evaporation can be controlled and adjusted by changing the flow rate of the blood and/or the gas. Such control may be applied using appropriate sensors, for osmolarity, flow, etc. located at a downstream end of the blood pathway (e.g., before the blood is returned to the patient's body). Control may be accomplished, e.g., using a needle valve or other regulator to adjust the rate at which gas exits a gas supply. The flow velocity of the gas, in turn, affects the evaporation rate of the water and hence the level of water removal. In alternative embodiments, the amount of water that is removed by evaporation can be controlled by varying the total surface area over which transfer of water molecules to the gas takes place (e.g. by providing multiple banks that each contain BFCs, and controlling the number of banks through which the blood and gas is routed).

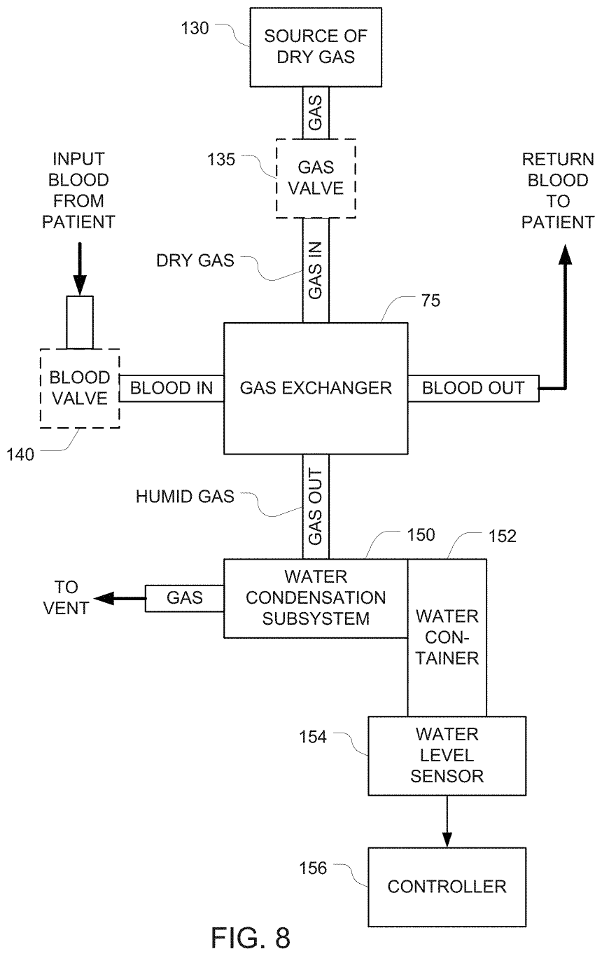

In some embodiments, the amount of water that is removed from the blood by evaporation is measured directly, and the measured quantity of water may be used to control the system (e.g., by turning off the system once a predetermined quantity of water has been removed). FIG. 8 depicts an example of a system that uses this direct measurement approach. The gas exchanger 75 in this embodiment may be constructed using any of the approaches described above. A gas (e.g., air, oxygen, etc.) from a source 130 flows into the input of the gas exchanger 75 via a valve 135, and then exits the gas exchanger. Meanwhile, blood from the patient enters the gas exchanger 75 via a valve 140, and then exits the gas exchanger and is returned to the patient. As the blood flows through the gas exchanger 75, water from the blood will evaporate into the gas. As a result, the gas that exits the gas exchanger 75 will be humid.

The gas that exits the gas exchanger 75 enters a water condensation subsystem 150, where the water vapor that was picked up in the gas exchanger 75 condenses into liquid water. A wide variety of conventional approaches for implementing the water condensation subsystem 150 will be apparent to persons skilled in the relevant arts, including but not limited to passing the humid gas over a cold surface with a high surface area. Moisture in the gas will condense into liquid water on the cold surface and is collected in a water container 152, and the water level in the water container 152 is measured by a water level sensor 154. The water level sensor 154 may be implemented using a wide variety of conventional approaches that will be apparent to persons skilled in the relevant arts, including but not limited to optical sensors, resistivity sensors, etc. The controller 156 receives a signal indicative of the water level from the water level sensor 154. When the amount of collected water reaches a set level, the controller 156 can stop the extraction of water from the blood by sending inappropriate signal to an actuator (not shown) that controls the gas valve 135 and/or an actuator (not shown) that controls the blood valve 140. Alternatively or additionally, the controller 156 can output an indication to healthcare personnel to disconnect the system or take another appropriate action.

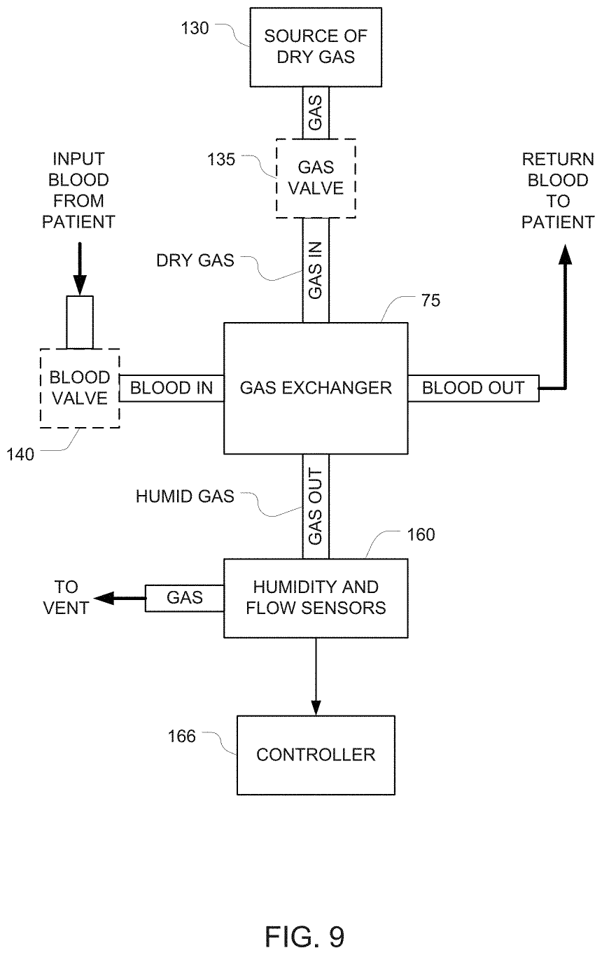

The amount of water that has been removed from the blood by evaporation may also be determined indirectly, e.g., by tracking the humidity of the gas that exits the gas exchange unit over time, and estimating the amount of water that is been removed based on the tracked level of humidity. In this case, the estimated quantity of water that has been removed is used to control the system. FIG. 9 depicts an example of a system that uses this indirect measurement approach. Similar to the FIG. 8 embodiment described above, as the blood flows through the gas exchanger 75, water from the blood will evaporate into the gas. As a result, the gas that exits the gas exchanger 75 will be humid. Humidity and flow sensors 160 measure the humidity and the flow the gas that is exiting the gas exchanger 75, and the time-varying humidity and time-varying flow data is provided to the controller 166. Based on these two time-varying data streams, the controller 166 can estimate the amount of water that is being exhausted via the vent at any given instant. The controller 166 keeps a running total of how much water has been exhausted. When the controller determines that the desired quantity of water has been removed, the controller 166 proceeds in the same way as the controller 156 (as described above in connection with FIG. 8).

Note that while the fluid reducer embodiments are described above in the context of removing water from blood, these same embodiments may also be used to remove other solvents from other liquids. This can be accomplished by routing the other liquid from left to right through the gas exchanger 75 and routing gas from top to bottom through the gas exchanger 75 in either FIG. 8 or FIG. 9; and subsequently measuring a level of condensed liquid in a container or concentration and flow in the exhaust gas, similar to the approach described above in connection with FIG. 8 and FIG. 9.

Optionally, these Fluid Reducer embodiments can also heat the blood to a desired temperature. One example of a suitable configuration that may be used to heat is to coat both top and bottom surfaces, except for the openings of the BFCs 2, by a thin coating layer, which is preferably made from an electrically conducting material such as carbon. Such coating layer (not shown) may be made, for example, by vapor deposition, which leaves the BFCs 2 open. This arrangement is particularly suited to those embodiments where the plurality of NTs is arranged like a carpet-like field with the voids therein forming the fluid-flow channels (as depicted in FIG. 3B).

Because carbon NTs are conductive and a large number of carbon NTs span the distance between the upper and lower coating layers, heating in this embodiment can be accomplished by applying a voltage between the upper and lower coating layers via conductive leads. The applied voltage will cause a current to flow through the NTs, which will generate heat. As the electric resistance between the upper and lower surfaces of a typical NT carpet-like field is about 10 Ω per an area of 1 cm², a 10 cm² carpet-like field would function as a 1 Watt heater when activated by a 1 Volt potential difference. Changing the voltage will change the amount of heat that is generated. The amount of heat that is added to the system can be controlled by controlling the voltage that is applied to the leads or by controlling the current that passes through those leads.

In certain circumstances, it may be beneficial to remove certain dissolved gases from liquids such as water, or blood without affecting the nature of other materials dissolved or suspended in the liquid. For example, in patients suffering from renal insufficiency, it may be desirable to remove harmful dissolved gases (e.g., ammonia) from the patient's blood.

A third set of embodiments is directed to removing gasses (e.g., ammonia) dissolved in the blood by allowing its efficient diffusion into the gas or air that is brought into contact with the blood. The hardware configuration of these embodiments is similar to the hardware configuration of the Fluid Reducer system described above (i.e., the second set of embodiments

When ammonia gas is dissolved in water, some of it converts to ammonium ions:

H₂O+NH₃ [symbol] OH⁻+NH₄⁺ (Equation No. 1)

Rapid and efficient elimination of ammonia (which is produced by protein degradation in living cells) from the blood will result in negligible concentrations of ammonia and ammonium in the blood and thus the quantity of urea synthesis by the liver will be markedly reduced. As the ammonia concentration is lowered the balance of equation (1) is moved to the left such that the ammonium is transformed to ammonia and is eliminated.

Effective removal is achieved by providing an extremely large area of contact between the blood and the gas. As in the Fluid Reducer embodiments, the NTs surrounding the BFC 2 form a virtual boundary for the blood flow. The interaction between the blood and the gas the diffusion of ammonia from the blood into the gas) occurs at this virtual boundary. When the blood flows in the BFCs it is in direct contact with the flowing gas such that the ammonia gas dissolved in the blood diffuses out to the flowing gas that contains no such molecules. The ammonia is carried away with the flowing gas. The blood ammonium and ammonia content is thus reduced. One can remove all the toxic material flowing in the blood and thus significantly reducing the amount of urea produced by the liver such that the amount of toxic material in the blood due to kidney failure is minimal.

The techniques described herein are not limited to removing ammonia from blood, and these same techniques can be used to remove any volatile molecule of interest from blood. More specifically, when blood is flowing through the BFCs 2, volatile molecules within the blood will diffuse out of the blood and into the flowing gas that originally contains no such molecules. This phenomenon can advantageously be utilized as a “diagnostic nose” by capturing the gas that exits the GEU and analyzing that gas to detect the presence and/or concentration of the molecules of interest. A diagnostic decision can then be made based on the detection and/or the concentration of the molecules of interest (or the absence of detected molecules).

Because there is direct blood-air contact, this type of “diagnostic nose” is superior to conventional artificial nose systems that rely on capturing air that is been exhaled from a person's lungs, and analyzing that exhaled air. This is because in the case of exhalation from the lungs, the relevant molecules in the blood must permeate the capillary and alveolar membranes before they can be detected. In contrast, the embodiments described herein provide direct blood-air contact, which increases the probability that the relevant molecule will be detected. The probability of detection is further increased because large quantities of blood will pass through the GEU.

Notably, the same hardware described above can also be used to introduce a specific molecule into a person's blood. Examples include introducing an anesthetic prior to surgery, introducing a pain killing compound, or adding various gases used for specific treatments (including but not limited to introducing CO₂ to control blood pH, minute amounts of CO to affect Hb function, etc.). In these embodiments, the relevant molecules are added (in gas form) to the gas before the gas enters the GEU. When the relevant molecules come into contact with the blood (at the virtual boundary of the channels), the molecules will diffuse into the blood, where they can perform their intended function (e.g., anesthesia, pain relief, therapeutic treatment, etc.).

Furthermore, the techniques described herein are not limited to removing molecules that are dissolved in blood, detecting molecules that are present in blood, and adding molecules in controlled doses to blood. To the contrary, the techniques described herein can be used to remove molecules from any other liquid, detect molecules that are present in any other liquid, and add molecules in controlled amounts to any other liquid.

The embodiments above are described in the context of delivering O₂ to blood and removing CO₂ from blood, removing excess water from blood, and removing toxins from blood. But the invention is not limited to those contexts, and can be used to deliver other gases to blood, or remove other components from blood. For example, it may be used in connection with a body part that has a dedicated circulation (such as a leg, brain, kidney) to deliver any desired gas to that body part. This can be used to deliver a chemical such as an anesthetic or therapeutic gas intended to act locally. In such a case the gas will be inputted into the artery and outputted (eliminated) via the vein, etc.

Note also that the invention is not limited to medical uses, and can be used to exchange gases in other types of fluid flow systems, including industrial applications.

As additional use of the apparatuses described above is as a heat exchanger. Regardless of whether any gases are exchanged between the gas and liquid that flow through the device, heat transfer can still occur between the gas and the fluid. As a result, hot fluid can be used to heat the gas, cold fluid can be used to cool the gas, hot gas can be used to heat the fluid, or cold gas can be used to cool the fluid. The heat transfer is expected to be very effective relative to prior art devices because the contact surface area is very large, and there is no physical barrier between the gas and the fluid. Optionally, sensors and pumps may be used to control the exchange so as to maintain the desired temperature. These sensors and pumps may also be used when the primary purpose is gas exchange, as in the embodiments described above.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

I claim:

1. A method for removing excess water from blood, the method comprising:
providing a plurality of fluid flow channels that are surrounded by hydrophobic nanotubes with diameters between 1 and 100 nm, with spaces between the nanotubes, each of the channels having an outer boundary, an inflow end, and an outflow end, wherein each of the channels is wide enough for blood to flow through, and wherein the nanotubes are spaced close enough to each other to retain the blood within the channels when the blood is flowing through the channels;
passing the blood through the channels; and
passing a gas through the spaces between the nanotubes outside the channels so that the gas comes into contact with the blood at the outer boundaries of the channels until the excess water in the blood evaporates into the gas.

2. The method of claim 1, further comprising:
determining whether a sufficient amount of water has been removed; and
discontinuing the passing of the blood after a sufficient amount of water has been removed.

3. The method of claim 1, wherein the nanotubes are carbon nanotubes.

4. The method of claim 1, wherein each of the channels has a diameter between 2 and 500 μm.

5. The method of claim 1, wherein the nanotubes have a diameter between 5 and 20 nm.

6. The method of claim 1, wherein the nanotubes are spaced on centers that are between 1.5 times the diameter of the nanotubes and 5 times the diameter of the nanotubes.

7. A method for removing excess solvent from a liquid, the method comprising:
providing a plurality of fluid flow channels that are surrounded by hydrophobic nanotubes with diameters between 1 and 100 nm, with spaces between the nanotubes, each of the channels having an outer boundary, an inflow end, and an outflow end, wherein each of the channels is wide enough for liquid to flow through, and wherein the nanotubes are spaced close enough to each other to retain the liquid within the channels when the liquid is flowing through the channels;
passing the liquid through the channels; and
passing a gas through the spaces between the nanotubes outside the channels so that the gas comes into contact with the liquid at the outer boundaries of the channels until the excess solvent in the liquid evaporates into the gas.

8. The method of claim 7, further comprising:
determining whether a sufficient amount of solvent has been removed; and
discontinuing the passing of the liquid after a sufficient amount of solvent has been removed.

9. A solvent evaporation apparatus, comprising:
a field of at least one million hydrophobic nanotubes with diameters between 1 and 100 nm with spaces between the nanotubes though which gas can travel, with voids in the field positioned to form a plurality of fluid flow channels, each of which is surrounded by the nanotubes, wherein the channels are wide enough for a liquid to pass through, and wherein the nanotubes adjacent to the channels are spaced close enough to each other to prevent the liquid from escaping the channels;
a gas pathway that passes through spaces between the nanotubes and extends from an input to the field of nanotubes to an output from the field of nanotubes;
at least one sensor that generates data indicative of how much solvent has been removed from the liquid; and
a controller that processes the data from the at least one sensor.

10. The apparatus of claim 9, wherein the liquid comprises blood and the solvent comprises water.

11. The apparatus of claim 10, further comprising a surface upon which the water condenses and a container for holding the condensed water, wherein the at least one sensor comprises a water level sensor that generates data indicative of how much water is in the container.

12. The apparatus of claim 10, wherein the at least one sensor comprises (a) a humidity sensor that outputs a first signal indicative of humidity of gas exiting the gas pathway and (b) a flow sensor that outputs a second signal indicative of flow of gas exiting the gas pathway, and wherein the controller determines how much water has exited the gas pathway based on the first signal and the second signal.
